(19) United States
(12) Patent Application Publication
(10) Pub. No.: US 2009/0292231 A1
(43) Pub. Date: Nov. 26, 2009

(54) RA COMFORT WRIST SUPPORT

(76) Inventor: REBECCA L. ELIAS, Pine Valley, CA (US)

Correspondence Address: WALTER A. HACKLER, Ph.D.;PATENT LAW OFFICE, 2327 S.E. BRISTOL STREET, SUITE B, NEWPORT BEACH, CA, 92660-0755, US

(21) Appl. No.: 12/123,881

(22) Filed: May 20, 2008

(51) Int. Cl. A61F 5/00

(52) U.S. Cl. 602/21; 602/6

(57) ABSTRACT

A splint assembly includes a radial correction hand device for correcting ulna finger deviation and a wrist support which includes a palm portion detachable to a palm side of the correction hand device and a ulna portion securable to a user's forearm.

The present invention generally relates to the field of wrist supports and more particularly is directed to a splint assembly for use in medical therapy and still more particularly to devices for treating rheumatoid arthritic hands.

Rheumatoid arthritis (RA) is a chronic systemic disease marked by inflammatory changes in the synovial membranes and articuler structures and by atrophy and rarefaction for bones.

Devices have been developed for correcting ulna finger deviation in a rheumatoid arthritic hand in which fingers are forced or pulled away from the thumb toward the little finger by the ulna muscles of the fingers.

U.S. Pat. No. 6,618,624 to Elias is such device and is to be incorporated herein in its entirety for a detailed description of how to make and use this device.

The Elias device, however, lacks a wrists support and cannot maintain a “functional position” of the wrist and hand for proper healing in the case of joint replacement surgery or proper positioning for pain relief.

The present invention provides an assembly which includes a radial correction hand device for correcting ulna finger deviation while at the same time providing functional positioning of a user's wrists.

### SUMMARY OF THE INVENTION

An RA comfort wrist support in accordance with the present invention generally includes a radial correction hand device for correcting ulna finger deviation in combination with a wrist support which includes a palm portion attached to a palm side of the correction hand device and ulna portion securable to a user's forearm with the wrist support including a length spanning the user's wrist.

More particularly, the wrist support palm portion is narrower than the wrist support ulna portion and preferably the wrist portion includes a moldable core which allows for accommodation depending on the patient/user's need. Preferably, the core may be molded to support the user's wrist in a functional position, for example, a position in which the user's wrist is extended back approximately 35 degrees for the hand to function properly and easily without injury.

The moldable core is preferably sandwiched between two slices of foam and at least one strap is provided for securing the wrist portion ulna portion to the user's forearm.

### BRIEF DESCRIPTION OF THE DRAWINGS

The present invention can be more readily understood by consideration of the following detailed description when taken in conjunction with the accompanying drawings, in which:

FIG. 1 is a side view of the present invention generally showing a radial correction hand device along with a wrist support disposed for maintaining a functional position of the wrist; and

FIG. 2 is a perspective view of the wrist support in accordance with the present invention partially broken away to show a multiple core sandwiched between slices of foam.

### DETAILED DESCRIPTION

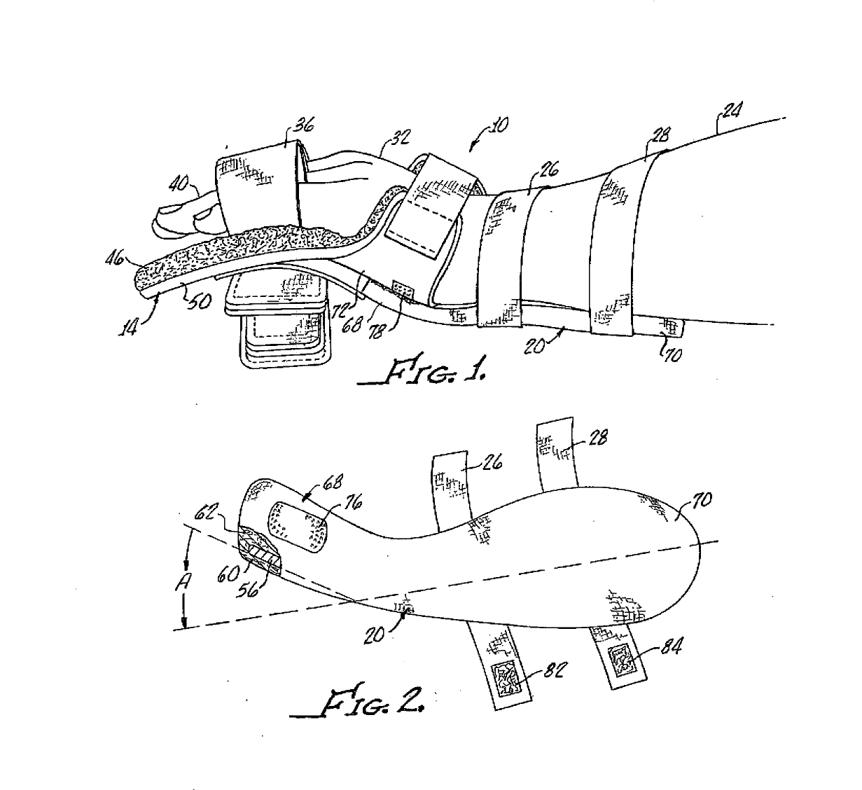

With reference to FIG. 1, there is shown an RA comfort wrist support 10 in accordance with the present invention which includes a radial collection hand device 14 and a wrist support 20 shown attached to a user's forearm 24 by means of straps 26, 28. The hand device 14 is depicted is applied to the user's hand 32 with finger straps 36 engaging fingers 40 to pull ulna deviated fingers back to their normal position (not shown). As shown, the hand 32 is disposed on a soft upper layer 46 of a hand-supporting pad 50.

A full description of a suitable hand device 14 may be found in U.S. Pat. No. 6,618,624 which is incorporated herewith for providing how to make and use the hand device 14.

With further reference to FIG. 2, the support 20 may be formed from a moldable aluminum core 56 sandwiched between slices 60, 62 of foam. As hereinabove noted, the moldable core may be angulated as shown in FIG. 2 to maintain a functional position of the user's hand 32 as shown in FIG. 1. This functional position is provided by angle A which is about 35 degrees between a palm portion 68 of the wrist support 20 and an ulna portion 70 of the wrist support 20. As illustrated, the palm portion 68 is narrower than the ulna portion 70.

The wrist support 20 may be permanently attached to the hand device 14, however, preferably the wrist support 20 is attachable to a palm side 72 of the hand device 14 by way of hooks 76 and loops 78 disposed on the wrist support 20 and hand device 14 respectively. Specifically, the hooks 76 and loops 78 may be a Velcro® type attachment. Similar Velcro® type attachment devices 82, 84 may be utilized to secure the straps 26, 28 about the user's forearm 24. A washable nylon cover (not shown) for the wrist support 20 may be provided.

Although there has been hereinabove described a specific splint assembly in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. An RA comfort wrist support comprising:
a radial correction hand device for correcting ulna finger deviation; and
a wrist support comprising a palm portion attachable to a palm side of the correction hand device, an ulna portion securable to a user's forearm and a length spanning a user's wrist.

2. An RA comfort wrist support assembly according to claim 1 wherein the wrist support palm portion is narrower than the wrist support ulna portion.

3. An RA comfort wrist support assembly according to claim 1 wherein the wrist support includes a moldable core.

4. An RA comfort wrist support assembly according to claim 3 wherein said moldable core is sandwiched between two slices of foam.

5. An RA comfort wrist support assembly according to claim 4 wherein the core is molded to support the user's wrist in a functional position.

6. An RA comfort wrist support assembly according to claim 5 wherein the functional position provides for the user's wrist to be extended back approximately 35 degrees.

7. An RA comfort wrist support assembly according to claim 1 further comprising hooks and loops for attaching the wrist support palm portion to the hand device palm side.

8. An RA comfort wrist support assembly according to claim 7 further comprising at least one strap for securing the wrist support ulna portion to the user's forearm.

9. An RA comfort wrist support comprising:
a radial correction hand device for correcting ulna finger deviation; and
a wrist support comprising a palm portion attached to a palm side of the correction hand device, an ulna portion securable to a user's forearm and a length spanning a user's wrist.

10. An RA comfort wrist support assembly according to claim 9 wherein the wrist support palm portion is narrower than the wrist support ulna portion.

11. An RA comfort wrist support assembly according to claim 9 wherein the wrist support includes a moldable core.

12. An RA comfort wrist support assembly according to claim 11 wherein said moldable core is sandwiched between two slices of foam.

13. An RA comfort wrist support assembly according to claim 12 wherein the core is molded to support the user's wrist in a functional position.

14. An RA comfort wrist support assembly according to claim 13 wherein the functional position provides for the user's wrist to be extended back approximately 35 degrees.

15. An RA comfort wrist support assembly according to claim 9 further comprising at least one strap for securing the wrist support ulna portion to the user's forearm.

16. An RA comfort wrist support assembly according to claim 15 wherein the wrist support palm portion is narrower than the wrist support ulna portion.

17. An RA comfort wrist support assembly according to claim 16 wherein the wrist support includes a moldable core.

18. An RA comfort wrist support assembly according to claim 17 wherein said moldable core is sandwiched between two slices of foam.

19. An RA comfort wrist support assembly according to claim 18 wherein the core is molded to support the user's wrist in a functional position.

20. An RA comfort wrist support assembly according to claim 5 wherein the functional position provides for the user's wrist to be extended back approximately 35 degrees.
